(19) United States
(12) Patent Application Publication
(10) Pub. No.: US 2024/0374256 A1
(43) Pub. Date: Nov. 14, 2024

(54) SURGICAL STAPLING DEVICE WITH JAWS THAT PIVOT BETWEEN AN OPEN POSITION AND A CLAMPED POSITION

(71) Applicant: Covidien LP, Mansfield, MA (US)

(72) Inventors: Roanit A. Fernandes, Hamden, CT (US); Matthew J. Chowaniec, Madison, CT (US)

(21) Appl. No.: 18/653,347

(22) Filed: May 2, 2024

Related U.S. Application Data
(60) Provisional application No. 63/464,992, filed on May 9, 2023.

(51) Int. Cl. A61B 17/072; A61B 17/29

(52) U.S. Cl. CPC A61B 17/07207; A61B 2017/07271; A61B 2017/2929

(57) ABSTRACT

A surgical stapling device includes a drive assembly and a tool assembly having an anvil and a cartridge assembly that is pivotably coupled to the anvil for movement between an open position and a clamped position. The cartridge assembly includes a staple cartridge and a channel member that defines a cavity that receives the staple cartridge. The channel member includes a channel lock that is positioned on a proximal portion of the channel member. The drive assembly is movable between a drive advanced position and a drive retracted position and engages the channel lock in the drive retracted position to retain the tool assembly in the open position.

### CROSS-REFERENCE TO RELATED APPLICATIONS

This application claims the benefit of U.S. Provisional Patent Application Ser. No. 63/464,992, filed May 9, 2023, the entire content of which is incorporated herein by reference.

### FIELD

This disclosure is directed to surgical stapling devices, and more particularly, to surgical stapling devices including jaws that pivot between an open position and a clamped position.

### BACKGROUND

Various types of surgical devices to endoscopically treat tissue are known in the art, and are commonly used, for example, for closure of tissue or organs in transection, resection, and anastomoses procedures, for occlusion of organs in thoracic and abdominal procedures, and for electrosurgically fusing or scaling tissue.

One example of such a surgical device is a stapling device. The stapling device includes an adapter assembly and a tool assembly supported on a distal portion of the adapter assembly. The tool assembly includes a first jaw that supports an anvil and a second jaw that supports a cartridge assembly. In some stapling devices, the anvil is fixedly supported on the anvil, or a mounting structure secured to the anvil, and the cartridge assembly is pivotally supported on the anvil, or a mounting structure secured to the anvil, such that the cartridge assembly can pivot in relation to the anvil between an open position and a clamped position. In some devices, the cartridge assembly is urged towards the open position by a biasing member.

Stapling devices configured for endoscopic or laparoscopic use are also known in the art. Such stapling devices are inserted through small incisions in the skin of a patient, or through small diameter cannulas, to access a surgical site within the patient. Typically, a clinician will use a tapered end of the cartridge assembly when the tool assembly is in the open position to dissect tissue within the body of the patient to access the surgical site. Since the cartridge assembly is not fixedly secured to the adapter assembly during the dissection of tissue, the cartridge assembly may not perform as desired.

A continuing need exists in the art for a stapling device that includes a cartridge assembly that can pivot in relation to the anvil between open and clamped positions while providing a rigid structure for dissecting tissue during positioning of the tool assembly at the surgical site.

### SUMMARY

This application is directed to a surgical stapling device that includes an adapter assembly, a tool assembly supported on the distal portion of the adapter assembly, and a drive assembly that is movable in relation to the tool assembly to actuate the tool assembly. The tool assembly includes an anvil and a cartridge assembly that is pivotably coupled to the anvil for movement between an open position and a clamped position. The cartridge assembly includes a staple cartridge and a channel member that defines a cavity that receives the staple cartridge. The channel member supports a channel lock that is positioned on a proximal portion of the channel member. The drive assembly is movable from a drive advanced position to a drive retracted position and engages the channel lock in the drive retracted position to retain the tool assembly in the open position.

One aspect of this disclosure is directed to a tool assembly including an anvil, a cartridge assembly, and a drive assembly. The cartridge assembly includes a channel member, a staple cartridge, and a channel lock. The channel member is pivotably coupled to the anvil and movable in relation to the anvil between open and clamped positions. The channel member includes a proximal portion and has side walls and a bottom wall. The bottom wall defines a first knife slot and extends between the side walls to define a cavity. The staple cartridge is receivable within the cavity and includes a cartridge body, staples, and an actuation sled. The cartridge body defines a second knife slot and staple receiving slots positioned on opposite sides of the second knife slot. The actuation sled is movable within the cartridge body from a sled advanced position to a sled retracted position to eject the staples from the cartridge body. The channel lock is secured to the proximal portion of the channel member. The drive assembly includes a drive member and a working end. The drive member is movable between a drive retracted position and a drive advanced position to move the actuation sled from the sled retracted position to the sled advanced position. In the drive retracted position, the drive assembly is engaged with the channel lock to retain the channel member of the cartridge assembly in the open position.

In aspects of the disclosure, the channel lock is secured to the bottom wall of the channel member and extends between the side walls of the channel member.

In some aspects of the disclosure, the working end of the drive assembly has an I-beam configuration and includes a first beam, a second beam, and a strut interconnecting the first beam and the second beam.

In certain aspects of the disclosure, the first beam is positioned to engage the anvil and the second beam is positioned to engage the cartridge assembly when the drive assembly is moved from the drive retracted position towards the drive advanced position.

In aspects of the disclosure, the second beam is positioned to engage the channel lock when the drive assembly is in the drive retracted position.

In some aspects of the disclosure, the vertical strut includes a cutting blade.

In certain aspects of the disclosure, the channel lock has a circular cross-section and is secured to the bottom wall of the channel member.

In aspects of the disclosure, the channel lock has a linear portion with bent ends, and the channel member defines openings that receive the bent ends of the channel lock.

In some aspects of the disclosure, the bottom wall of the channel member includes a proximal portion that defines a cutout that communicates with the first knife slot.

In certain aspects of the disclosure, the working member of the drive assembly is received within the cutout when the drive assembly is in the drive retracted position.

In aspects of the disclosure, the channel lock is positioned proximally of the working member when the drive assembly is in the drive retracted position.

Other aspects of the disclosure are directed to a cartridge assembly including a channel member and a staple cartridge. The channel member includes a proximal portion and has side walls and a bottom wall. The bottom wall defines a first knife slot and extends between the side walls to define a cavity. The staple cartridge is receivable within the cavity and includes a cartridge body, staples, and an actuation sled. The cartridge body defines a second knife slot and staple receiving slots positioned on opposite sides of the second knife slot. The actuation sled is movable within the cartridge body from a sled advanced position to a sled retracted position to eject the staples from the cartridge body. The channel lock is secured to the proximal portion of the channel member and extends between the side walls of the channel member.

Still other aspects of the disclosure are directed to a channel assembly of a cartridge assembly for a surgical stapling device, the channel assembly including a channel member and a channel lock. The channel member includes a proximal portion and has side walls and a bottom wall. The bottom wall defines a first knife slot and extends between the side walls to define a cavity. The channel lock is secured to the proximal portion of the channel member and extends between the side walls of the channel member.

Other features of the disclosure will be appreciated from the following description.

### BRIEF DESCRIPTION OF THE DRAWINGS

Various aspects of the disclosed surgical stapling device are described herein below with reference to the drawings, wherein:

FIG. 1 is a side perspective view of a surgical stapling device according to aspects of this disclosure with a tool assembly in an open position;

FIG. 2 is an enlarged view of the indicated area of detail shown in FIG. 1;

FIG. 2A is an exploded perspective view of a cartridge assembly of the tool assembly shown in FIG. 2;

FIG. 3 is an exploded view of a channel member of the cartridge assembly shown in FIG. 2A;

FIG. 4 is a side perspective view of a proximal portion of the channel member shown in FIG. 3;

FIG. 5 is a side perspective view of a distal portion of a drive assembly of the stapling device shown in FIG. 1;

FIG. 6 is a side perspective view of the tool assembly of the stapling device shown in FIG. 1 with a staple cartridge removed from the channel member of the cartridge assembly;

FIG. 7 is a side perspective view of the tool assembly of the stapling device shown in FIG. 1 as the drive assembly is moved from a drive advanced position back to the drive retracted position after the stapling device is fired;

FIG. 8 is an enlarged view of the indicated area of detail shown in FIG. 7; and

FIG. 9 is a side perspective view of the tool assembly as the staple cartridge is inserted into the channel member of the cartridge assembly of the stapling device shown in FIG. 1.

### DETAILED DESCRIPTION

The disclosed surgical stapling device will now be described in detail with reference to the drawings in which like reference numerals designate identical or corresponding elements in each of the several views. However, it is to be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

As used herein, the term “distal” refers to the portion of the stapling device that is being described which is further from a user during use of the device in its customary manner, while the term “proximal” refers to the portion of the stapling device that is being described which is closer to a user during use of the device in its customary manner. In addition, directional terms such as front, rear, upper, lower, top, bottom, and similar terms are used to assist in understanding the description and are not intended to limit the disclosure, and the term “clinician” is used generally to refer to medical personnel including doctors, nurses, surgeons, and support personnel.

This application is directed to a surgical stapling device that includes an adapter assembly, a tool assembly supported on the distal portion of the adapter assembly, and a drive assembly that is movable in relation to the tool assembly to actuate the tool assembly. The tool assembly includes an anvil and a cartridge assembly that is pivotably coupled to the anvil for movement between an open position and a clamped position. The cartridge assembly includes a staple cartridge and a channel member that defines a cavity that receives the staple cartridge. The channel member supports a channel lock that is positioned on a proximal portion of the channel member. The drive assembly is movable from a drive advanced position to a drive retracted position and engages the channel lock in the drive retracted position to retain the tool assembly in the open position.

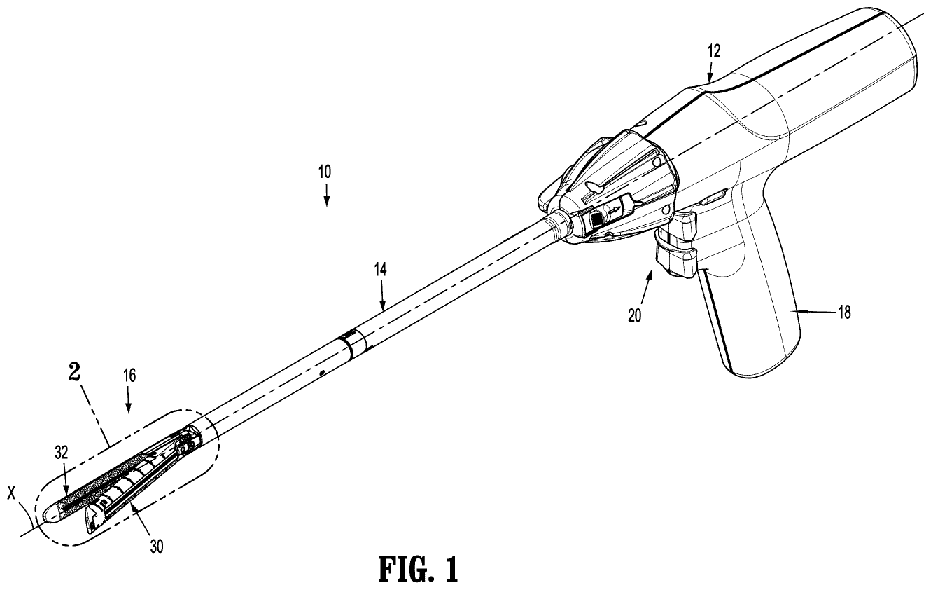

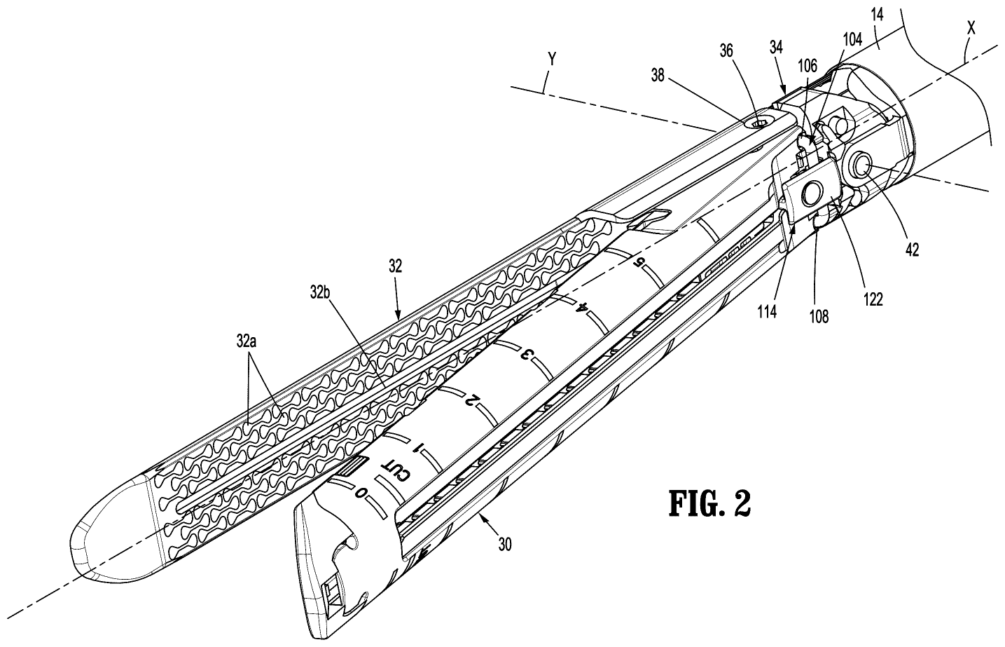

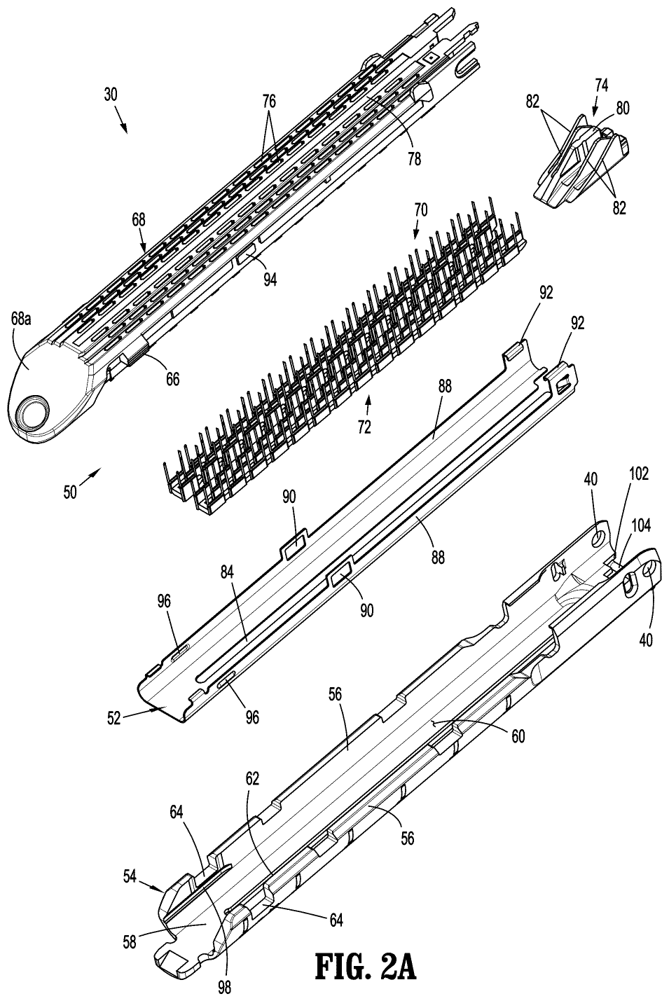

FIGS. 1 and 2 illustrate a surgical stapling device shown generally as stapling device 10 that includes a handle assembly 12, an elongate body or adapter assembly 14, and a tool assembly 16. As illustrated, the handle assembly 12 is powered and includes a stationary handgrip 18 and actuation buttons 20. The actuation buttons 20 are operable to actuate various functions of the tool assembly 16 via the adapter assembly 14 including approximation, stapling, cutting, and articulation. In certain aspects of the disclosure, the handle assembly 12 supports batteries (not shown) that provide power to the handle assembly 12 to operate the stapling device 10. Although the stapling device 10 is illustrated as a powered stapling device, it is envisioned that the features of this disclosure are suitable for use with manually powered surgical stapling devices as well as robotically controlled stapling devices.

The tool assembly 16 of the stapling device 10 includes a cartridge assembly 30 and an anvil 32. The anvil 32 includes a forming surface that defines staple forming pockets 32a and a knife slot 32b. In aspects of the disclosure, the tool assembly 16 is coupled to the adapter assembly 14 by a mounting assembly 34 (FIG. 2). In aspects of the disclosure, the proximal portion of the anvil 32 defines openings 38 (FIG. 2) and the proximal portion of the cartridge assembly 30 defines openings 40 (FIG. 2A). Screws 36 are received within the openings 38 of the anvil and in the mounting assembly 34 to fixedly secure the anvil 32 to the mounting assembly 34, and the screws 36 are received within the openings 40 of the cartridge assembly 30 to pivotably secure the cartridge assembly 30 to the mounting assembly 34 and the anvil 32 to facilitate movement of the tool assembly 16 between an open position and a clamped position.

In aspects of the disclosure, the mounting assembly 34 is secured to the distal portion of the adapter assembly 14 by a pivot member 42 that defines an articulation axis “Y” that is transverse to a longitudinal axis “X” (FIG. 1) defined by the adapter assembly 14 to pivotably secure the tool assembly 16 to the distal portion of the adapter assembly 14. It is envisioned that aspects of this disclosure are suitable for use with stapling devices that include non-articulating tool assemblies.

FIG. 2A illustrates the cartridge assembly 30 which includes a staple cartridge 50 having a staple guard 52, and a channel member 54. The channel member 54 includes side walls 56 and a bottom wall 58 that together define a cavity 60 that receives the staple cartridge 50. The bottom wall 58 of the channel member 54 defines a knife slot 62 that extends from the proximal end of the channel member 54 to a position near a distal end of the channel member 54. In aspects of the disclosure, the side walls 56 of the channel member include cut outs 64 that receive protrusions 66 formed on the staple cartridge 50 to properly position the staple cartridge 50 within the cavity 60 of the channel member 54.

The staple cartridge 50 includes a cartridge body 68, staples 70, pushers 72, and an actuation sled 74. The cartridge body 68 defines a plurality of staple receiving slots 76 and a knife slot 78 that is aligned with the knife slot 62 in the channel member 54 when the staple cartridge 50 is received within the cavity 60 of the channel member 54. A distal portion of the cartridge body 68 includes an angled surface 68a that defines a plane that extends away from the anvil 32 in a distal direction. In aspects of the disclosure, the staple receiving slots 76 are aligned in rows on opposite sides of the knife slot 78 although other arrays of staple receiving slots 76 are envisioned. Each of the staple receiving slots 76 receives one of the staples 70 and one of the pushers 72. The actuation sled 74 includes a central body portion 80 and cam members 82 that are positioned on opposite sides of the central body portion 80. The central body portion 80 is received within the knife slot 78 of the cartridge body 68 to guide movement of the actuation sled 74 through the cartridge body 68 from a sled retracted position to a sled advanced position. As the actuation sled 74 is moved from the sled retracted position towards the sled advanced position, the cam members 82 move into sequential engagement with the pushers 72 to eject the staples 70 from the cartridge body 68.

The staple guard 52 is secured to a bottom of the cartridge body 68 of the staple cartridge 50 to retain the staples 70 and the pushers 72 within the cartridge body 68 during shipping and handling of the staple cartridge 50. The staple guard 52 is positioned between the cartridge body 68 and the bottom wall 58 of the channel member 54 when the staple cartridge 50 is received within the cavity 60 of the channel member 54 and defines a knife slot 84 that is aligned with the knife slot 62 of the of the channel member 54 and the knife slot 78 of the cartridge body 68. In aspects of the disclosure, the staple guard 52 includes side walls 88 that define centrally located openings 90 and support proximally located hooks 92. The openings 90 receive protrusions 94 formed on the cartridge body 68 to secure the staple guard 52 to the cartridge body 68. Similarly, the hooks 92 engage a proximal portion of the cartridge body 68 to secure the staple guard 52 to the cartridge body 68. In some aspects of the disclosure, the staple guard 52 also includes distally located ribs 96 that extend outwardly from the side walls 88 of the staple guard 52 into channels 98 defined in the inner surfaces of the side walls 56 of the channel member 54 to releasably retain the staple cartridge 50 within the cavity 60 of the channel member 54. The staple cartridge 50 is releasably retained within the cavity 60 of the channel member 54 to facilitate replacement of the staple cartridge 50 and reuse the stapling device 10.

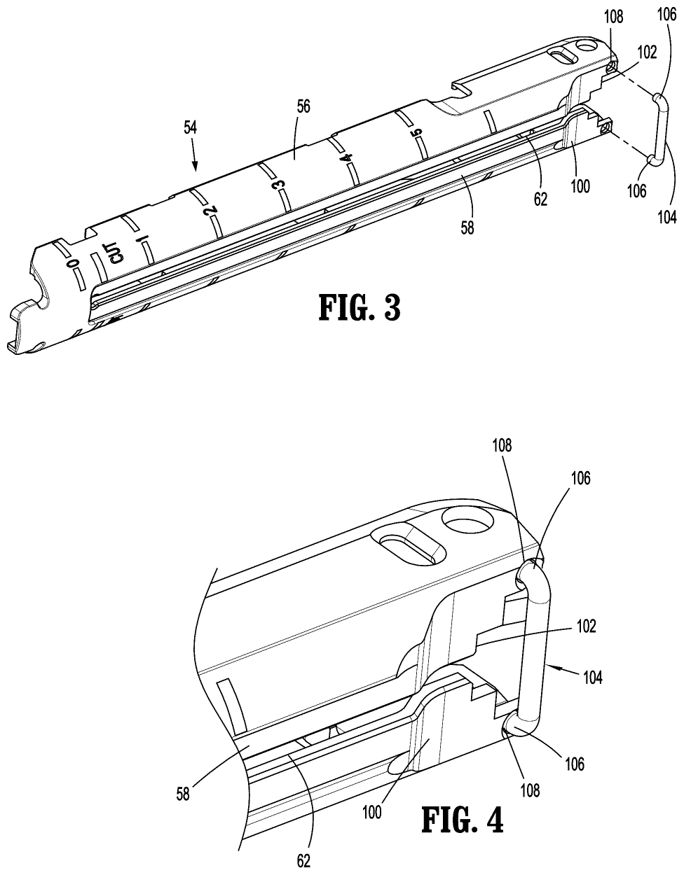

FIGS. 3 and 4 illustrate the channel member 54 of the cartridge assembly 30 (FIG. 2A) of the stapling device 10 (FIG. 1). The bottom wall 58 of the channel member 54 has an outer surface that defines a ramp 100 that is positioned on the proximal portion of the channel member 54. The bottom wall 58 also defines a cutout 102 that communicates with the knife slot 62 and is positioned proximally of the ramp 100. In aspects of the disclosure, the cutout 102 has a stepped rectangular configuration although other cutout configurations are envisioned. The proximal portion of the channel member 54 supports a channel lock 104 that extends between the side walls 56 of the channel member 54 across the proximal portion of the cutout 102. In aspects of the disclosure, the channel lock 104 has a linear portion with bent ends 106 that are secured within openings 108 formed in the bottom wall 58 of the channel member 54. In certain aspects of the disclosure, the bent ends 106 are welded to the bottom wall 58 of the channel member 54 to fixedly secure the channel lock 104 to the proximal end of the channel member 54. In certain aspects of the disclosure, the channel lock 104 has a circular cross-section although other cross-sectional shapes are envisioned.

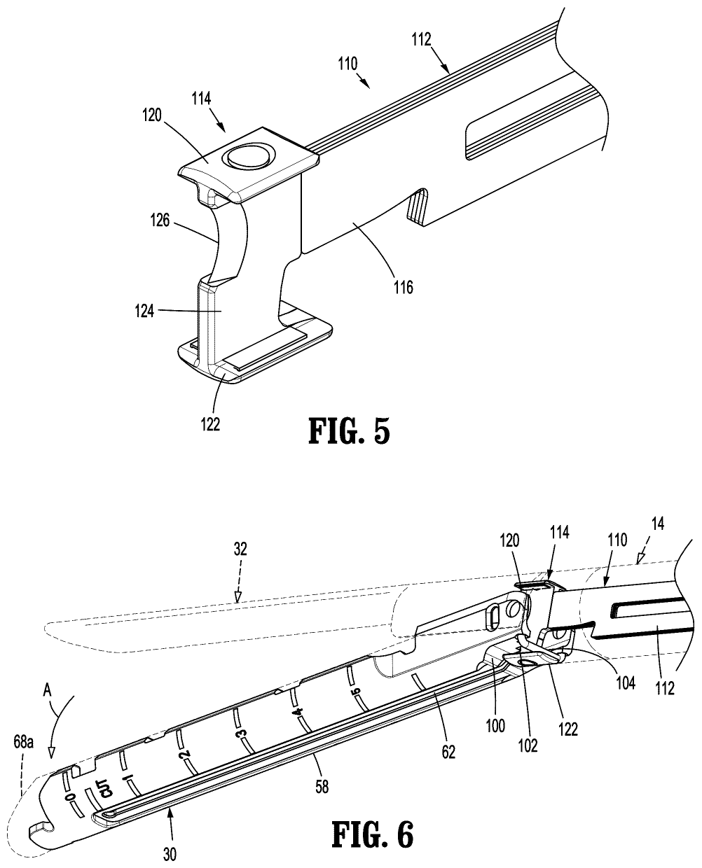

FIG. 5 illustrates a drive assembly 110 of the stapling device 10 (FIG. 1) which includes a drive member 112 and a working end 114. In aspects of the disclosure, the drive member 112 includes a proximal portion (not shown) and a distal portion 116. The proximal portion of the drive member 112 is coupled to a drive mechanism (not shown) within the adapter assembly 14 of the stapling device 10 (FIG. 1) such that longitudinal movement of the drive mechanism causes longitudinal movement of the drive assembly 110. The distal portion 116 of the drive member 112 is secured to the working end 114 of the drive assembly 110 such that longitudinal movement of the drive member 112 causes longitudinal movement of the working end 114 in relation to the tool assembly 16 between a drive retracted position and a drive advanced position. In the drive retracted position, the working end 114 of the drive assembly 110 is received in the cutout 102 in the proximal portion of the channel member 54. In aspects of the disclosure, the working end 114 of the drive assembly 110 is welded or pinned to the distal portion 116 of the drive member 112.

The working end 114 of the drive assembly 110 is rigid and has an I-beam configuration with a first beam 120, a second beam 122 and a vertical strut 124. The first beam 120 is secured to, or formed with, one end of the vertical strut 124 and the second beam 122 is secured to, or formed with, an opposite end of the vertical strut 124. In aspects of the disclosure, the vertical strut 124 includes or supports a cutting blade 126. When the drive assembly 110 is in the drive retracted position, the working end 114 of the drive assembly 110 is received within the cutout 102 of the channel member 54 with the vertical strut 124 aligned with the proximal end of the knife slot 62 defined in the channel member 54.

FIG. 6 illustrates the distal portion of the stapling device 10 (FIG. 1) with the drive assembly 110 in the drive retracted position. When the drive assembly 110 is the drive retracted position, the working end 114 of the drive assembly 110 is received in the cutout 102 in the proximal portion of the channel member 54 of the cartridge assembly 30 such that the second beam 122 is positioned proximally of the ramp 100 of the channel member 54 and engaged with the channel lock 104. When the second beam 122 of the working end 114 engages, or is engaged with, the channel lock 104, the working end 114 urges or retains the tool assembly 16 in the open position.

When the stapling device 10 (FIG. 1) is fired, the first beam 120 engages the anvil 32 and the second beam 122 engages the ramp 100 of the channel member 54 such that longitudinal movement of the working end 114 of the drive assembly 110 causes the tool assembly 16 (FIG. 1) to move between the open and clamped positions. The vertical strut 124 of the working end 114 of the drive assembly 110 is positioned proximally of and in alignment with the actuation sled 74 of the staple cartridge 50 such that advancement of the working end 114 of the drive assembly 110 in relation to the anvil 32 and the cartridge assembly 30 causes advancement of the actuation sled 74 within the cartridge body 68 of the staple cartridge 50. As the actuation sled 74 is moved from the sled retracted position towards the sled advanced position, the cam members 82 (FIG. 2A) of the actuation sled 74 move into sequential engagement with the pushers 72 to eject the staples 70 from the cartridge body 68.

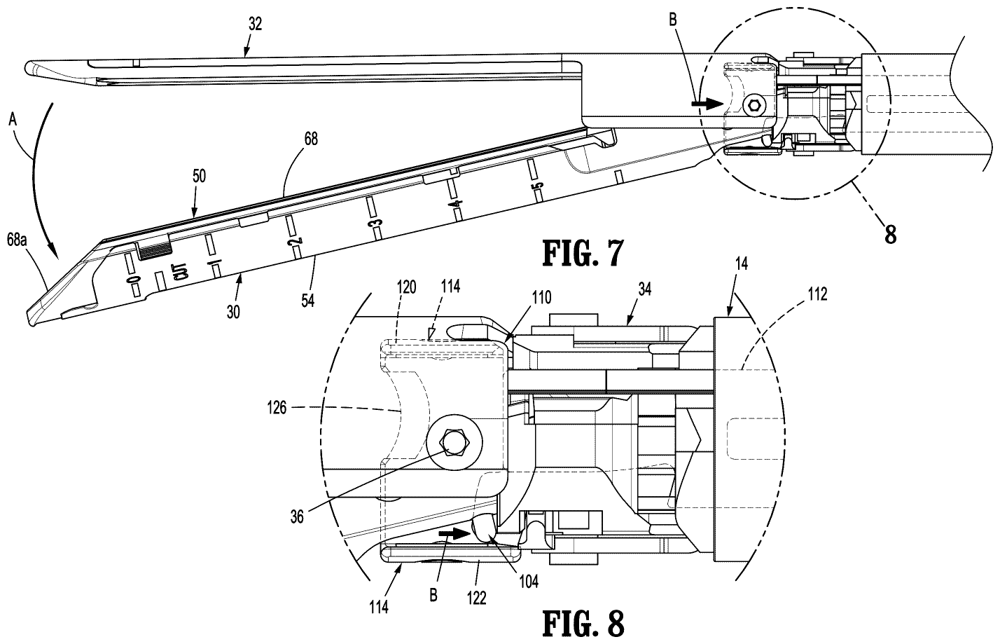

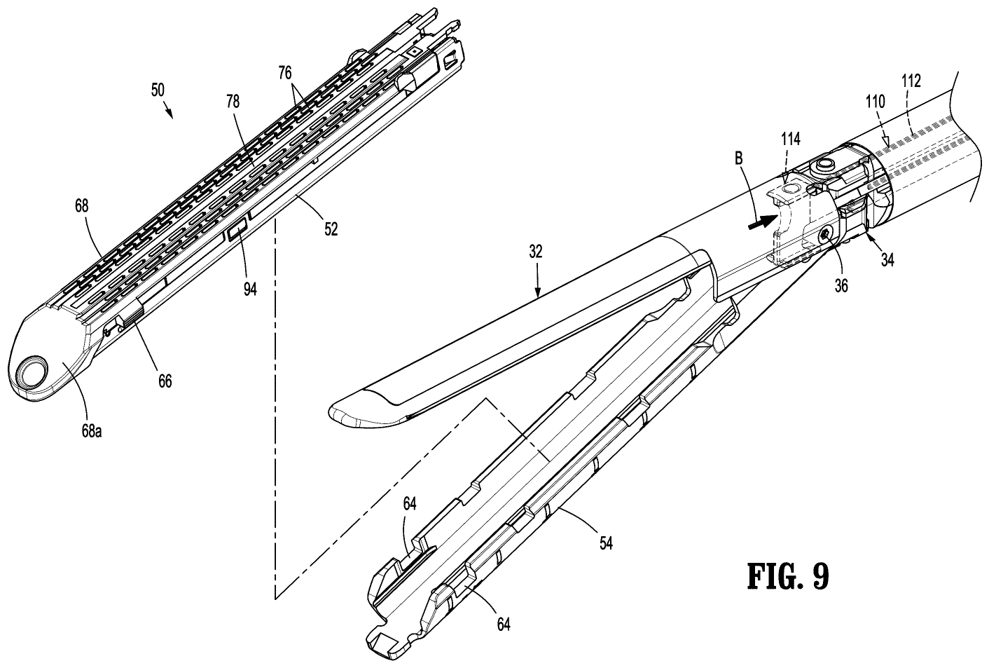

FIGS. 7-9 illustrate the tool assembly 16 of the stapling device 10 (FIG. 1) as the drive assembly 110 moves in the direction indicated by arrow “B” from the drive advanced position to the drive retracted position. After the stapling device 10 is fired and the drive assembly 110 is returned to the drive retracted position, the second beam 122 engages the channel lock 104 that is secured to the proximal end of the channel member 54 to pivot the channel member 54 and the staple cartridge 50 to the open position. In the open position, the staple cartridge 50 is easily accessible by a clinician to facilitate replacement of the staple cartridge 50 and reuse of the stapling device 10 (FIG. 9).

It is noted that during an endoscopic or laparoscopic surgical procedure, after the tool assembly 16 is inserted through a small diameter cannula (not shown), the tool assembly 16 can be moved to the open position to accommodate tissue prior to clamping the tissue between the anvil 32 and the cartridge assembly 30. When the tool assembly 16 is moved to the open position within a body cavity of a patient, the second beam 122 of the working end 114 of the drive assembly 110 engages the channel lock 104 to retain the cartridge assembly 30 in the open position. The channel lock 104 provides stability to the cartridge assembly 30 to allow the angled distal surface 68a of the cartridge body 68 to be used by the clinician to dissect tissue adjacent the surgical site and facilitate proper placement/positioning of the toll assembly about target tissue.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. Also, one skilled in the art will appreciate further features and advantages of the disclosure based on the above-described aspects. Accordingly, the disclosure is not to be limited by what has been particularly shown and described, except as indicated by the appended claims.

What is claimed is:

1. A tool assembly comprising:
an anvil;
a cartridge assembly including a channel member, a staple cartridge, and a channel lock, the channel member being pivotably coupled to the anvil and movable in relation to the anvil between open and clamped positions, the channel member including a proximal portion and having side walls and a bottom wall, the bottom wall defining a first knife slot and extending between the side walls to define a cavity, the staple cartridge receivable within the cavity and including a cartridge body, staples, and an actuation sled, the cartridge body defining a second knife slot and staple receiving slots positioned on opposite sides of the second knife slot, the actuation sled movable within the cartridge body from a sled advanced position to a sled retracted position to eject the staples from the cartridge body, the channel lock secured to the proximal portion of the channel member; and
a drive assembly including a drive member and a working end, the drive member movable between a drive retracted position and a drive advanced position to move the actuation sled from the sled retracted position to the sled advanced position, wherein in the drive retracted position, the drive assembly is engaged with the channel lock to retain the channel member of the cartridge assembly in the open position.

2. The tool assembly of claim 1, wherein the channel lock is secured to the bottom wall of the channel member and extends between the side walls of the channel member.

3. The tool assembly of claim 2, wherein the working end of the drive assembly has an I-beam configuration and includes a first beam, a second beam, and a strut interconnecting the first beam and the second beam.

4. The tool assembly of claim 3, wherein the first beam is positioned to engage the anvil and the second beam is positioned to engage the cartridge assembly when the drive assembly is moved from the drive retracted position towards the drive advanced position.

5. The tool assembly of claim 4, wherein the second beam is positioned to engage the channel lock when the drive assembly is in the drive retracted position.

6. The tool assembly of claim 5, wherein the vertical strut includes a cutting blade.

7. The tool assembly of claim 5, wherein the channel lock has a circular cross-section and is secured to the bottom wall of the channel member.

8. The tool assembly of claim 1, wherein the channel lock has a linear portion with bent ends, and the channel member defines openings that receive the bent ends of the channel lock.

9. The tool assembly of claim 1, wherein the bottom wall of the channel member includes a proximal portion defining a cutout that communicates with the first knife slot.

10. The tool assembly of claim 9, wherein the working member of the drive assembly is received within the cutout when the drive assembly is in the drive retracted position.

11. The tool assembly of claim 10, wherein the channel lock is positioned proximally of the working member when the drive assembly is in the drive retracted position.

12. A cartridge assembly comprising:
a channel member including a proximal portion and having side walls and a bottom wall, the bottom wall defining a first knife slot and extending between the side walls to define a cavity;
a staple cartridge receivable within the cavity and including a cartridge body, staples, and an actuation sled, the cartridge body defining a second knife slot and staple receiving slots positioned on opposite sides of the second knife slot, the actuation sled movable within the cartridge body from a sled advanced position to a sled retracted position to eject the staples from the cartridge body; and
a channel lock secured to the proximal portion of the channel member and extending between the side walls of the channel member.

13. The cartridge assembly of claim 12, wherein the channel lock has a circular cross-section and is secured to the bottom wall of the channel member.

14. The cartridge assembly of claim 12, wherein the bottom wall of the channel member includes a proximal portion defining a cutout that communicates with the first knife slot.

15. The cartridge assembly of claim 14, wherein the channel lock extends across the cutout between the side walls of the channel member.

16. The cartridge assembly of claim 12, wherein the channel lock has a linear portion with bent ends, and the channel member defines openings that receive the bent ends of the channel lock.

17. A channel assembly of a cartridge assembly for a surgical stapling device, the channel assembly comprising:
a channel member including a proximal portion and having side walls and a bottom wall, the bottom wall defining a first knife slot and extending between the side walls to define a cavity; and
a channel lock secured to the proximal portion of the channel member and extending between the side walls of the channel member.

18. The channel assembly of claim 17, wherein the channel lock has a circular cross-section and is secured to the bottom wall of the channel member.

19. The channel assembly of claim 17, wherein the bottom wall of the channel member includes a proximal portion defining a cutout that communicates with the first knife slot.

20. The channel assembly of claim 17, wherein the channel lock has a linear portion with bent ends, and the channel member defines openings that receive the bent ends of the channel lock.
